(19) United States
(12) Patent Application Publication
(10) Pub. No.: US 2002/0091256 A1
(43) Pub. Date: Jul. 11, 2002

(54) Azepine compounds

(76) Inventors: Masaru Matsuoka, Tenri-shi (JP); Kazuko Shirai, Osaka (JP); Toru Kitaguchi, Yokohama-shi (JP); Shigeki Kambara, Himeji-shi (JP)

Correspondence Address: BIRCH STEWART KOLASCH & BIRCH, PO BOX 747, FALLS CHURCH, VA, 22040-0747, US

(21) Appl. No.: 09/986,164

(22) Filed: Nov. 7, 2001

(30) Foreign Application Priority Data
Nov. 7, 2000 (JP) 339088/2000

(51) Int. Cl. C07D 487/02; C07D 243/08; C07D 243/10

(52) U.S. Cl. 540/567; 540/575; 540/476

(57) ABSTRACT

The azepine compound capable of emitting a light by light-irradiation or action of an electric field is represented by the following formula (I) or (II):

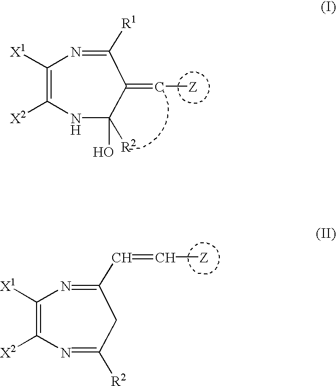

wherein X¹ and X² are the same or different, each representing an electron attractive group; R¹ represents a hydrogen atom, an alkyl group, an aryl group, an aralkyl group, or an alkoxy group; R² represents an amino group or an N-substituted amino group, or, in the formula (I), R² bonds a carbon atom adjacent to a ring Z to form a ring; and the ring Z represents a hydrocarbon ring which may have a substituent or a heterocycle which may have a substituent.

The compound is useful as a functional material, thus, an organic electroluminescence device can be obtained by interposing an organic layer composed of the above compound between a pair of electrodes.

### FIELD OF THE INVENTION

The present invention relates to novel azepine compounds useful as functional materials (particularly, materials for use in organic electroluminescence devices), a process for producing the same, and an organic electroluminescence device using the same.

### BACKGROUND OF THE INVENTION

As a fluorescent pigment, a compound having a planar structure and a hard or rigid π-conjugated system (e.g., stilbene, coumarin, naphthalimide, perylene, Rhodamine) is known. Meanwhile, a fluorescent pigment such as a compound having a pyrazine ring (e.g., styryl pyrazine, 2,5-bis(dialkylamino)-3,6-dicyanopyrazine, pyrazino heterocyclic compound, pyrazino phthalocyanine) is also known. Since these pigments not only emit fluorescent light upon irradiation of light but have such functions as light-absorptivity (e.g., color, pleochroism), photoconductivity, and reversible changes by heat or light (e.g., thermochromism, photochromism), these have been used as functional materials in a variety of fields (e.g., fluorescent materials, photochromic materials, optical recording materials). In particular, those that emit light by the action (application) of electric fields are useful as emission center compounds for use in organic electroluminescence devices (hereinafter, occasionally abbreviated as organic EL device) which is desired to be fully colored.

Conventionally, organic electroluminescence devices are composed of a compound or compounds having an electron-transporting function, a hole-transporting function, and an emission center function. There have been reported single-layered ones in which a single layer is provided with all the functions mentioned above, and multi-layered ones in which layers have different functions. Its principle of light emission is considered to be based on the phenomenon that electrons and holes injected from a pair of electrodes recombine within a light-emitting layer to form excitons, exciting an emission center compound constituting a light-emitting layer.

Colors that organic EL devices emit can be selected by suitably choosing an emission center compound constituting the light-emitting layer. For example, Japanese Patent Application Laid-Open No. 73443/1996 (JP-8-73443A) discloses the dimer of pyrazine in which a pyrazine group having a phenyl group is bound to a divalent aromatic group, and an organic EL device containing this pyrazine derivative. However, since this pyrazine dimer emits blue light of which the wavelength is relatively short, the electroluminescence device is limited in its emission wavelength and thus has greatly limited applications.

### SUMMARY OF THE INVENTION

Thus, it is an object of the present invention to provide a compound capable of emitting light upon irradiation of light or by the action of electric fields and useful as a functional material such as an organic EL device-use material, a process for producing the same, and an organic EL device using the same.

Another object of the present invention is to provide a compound of which the emission wavelength is controllable over a wide range and capable of emitting light of longer wavelength (e.g., yellow to red light), and an organic EL device using the same.

The inventors of the present invention made intensive studies to achieve the above objects and finally found that a compound, in which a specific ring is bonded to an azepine ring via the C═C double bond, emits light upon irradiation of light or by the action of electric fields and therefore is useful as such a functional material as those for organic electroluminescence devices. The present invention was accomplished based on the above findings.

That is, the azepine compound of the present invention is represented by the following formula (I) or (II):

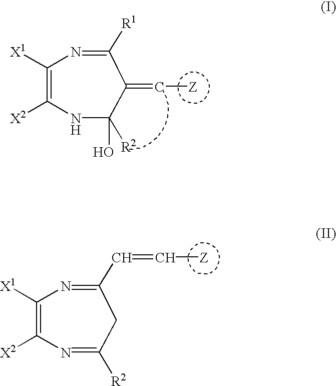

wherein X¹ and X² are the same or different, each representing an electron attractive group; R¹ represents a hydrogen atom, an alkyl group, an aryl group, an aralkyl group, or an alkoxy group; R² represents an amino group or an N-substituted amino group, or, in the formula (I), R² bonds a carbon atom adjacent to a ring Z to form a ring; and the ring Z represents a hydrocarbon ring which may have a substituent or a heterocycle which may have a substituent.

At least one of X¹ and X² may be a cyano group. R² may be an N- mono- or diC₁₋₄alkylamino group. R² may bond a carbon atom adjacent to the ring Z to form a 4- to 8-membered heterocycle. The hydrocarbon ring or the heterocycle of the ring Z may be an aromatic ring. Such an azepine compound is capable of emitting light by being irradiated with light or by the action of an electric field.

The present invention further includes a process for producing the above-mentioned compound (I) or (II).

The present invention further includes an organic electroluminescence device having, between a pair of electrodes, an organic layer (light-emitting layer) comprising a compound represented by the formula (I) or (II) shown above. The organic layer of this organic electroluminescence device may have (1) a single-layered structure composed of a light-emitting layer having at least one function selected from an electron-transporting function and a hole-transporting function, or (2) a multilayered structure (lamination) composed of a layer having at least one function selected from an electron-transporting function and a hole-transporting function, and a light-emitting layer.

### BRIEF DESCRIPTION OF THE DRAWINGS

FIG. 1 is an emission spectrum of the organic electroluminescence devices obtained in Example 3 and 4.

FIG. 2 is a graph showing the emission luminance value(cd/m²) of the organic electroluminescence devices obtained in Examples 3 and 4 versus voltage applied thereto.

### DETAILED DESCRIPTION OF THE INVENTION

An azepine compound of the present invention is shown by the following formula (I) or (II).

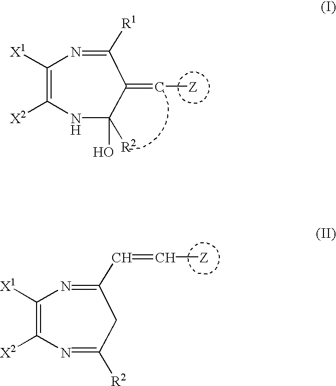

wherein X¹ and X² are the same or different, each representing an electron attractive (electron-withdrawing) group; R¹ represents a hydrogen atom, an alkyl group, an aryl group, an aralkyl group, or an alkoxy group; R² represents an amino group or an N-substituted amino group, or, in the formula (I), R² bonds a carbon atom adjacent to a ring Z to form a ring; and the ring Z represents a hydrocarbon ring which may have a substituent or a heterocycle which may have a substituent.

Exemplified as the electron attractive group represented by X¹ and X² are a cyano group, a carbonyl group. As the electron attractive group, the cyano group is preferred. Usually, at least one of X¹ and X² is the cyano group, and it is preferred that both of which are cyano groups. An azepine ring having such X¹ and X² seems to function as an acceptor upon intramolecular charge transfer.

Exemplified as the alkyl groups represented by R¹ are C₁₋₂₀ alkyl groups (e.g., C₁₋₁₀ alkyl groups, preferably C₁₋₆ alkyl groups, more preferably C₁₋₄ alkyl groups, particularly methyl and ethyl groups) such as methyl, ethyl, propyl, isopropyl, butyl, t-butyl, pentyl, hexyl, and octyl groups; as the aryl group are C₆₋₂₀ aryl groups (preferably C₆₋₁₈ aryl groups, more preferably C₆₋₁₄ aryl groups, particularly phenyl group) such as phenyl, naphthyl, and biphenyl groups; as the aralkyl group are C₇₋₂₀ aralkyl groups (e.g., C₆₋₁₂aryl-C₁₋₈alkyl groups, preferably C₆₋₁₂aryl-C₁₋₆alkyl groups, particularly benzyl group) such as benzyl and phenethyl groups; and as the alkoxy group are C₁₋₂₀alkoxy groups (preferably C₁₋₁₀alkoxy groups, more preferably C₁₋₆alkoxy groups) such as methoxy, ethoxy, propoxy, butoxy, and t-butoxy groups.

Exemplified as the N-substituted amino groups represented by R² are mono- or diC₁₋₆alkylamino groups, preferably mono- or diC₁₋₄alkylamino groups, more preferably mono- or diC₁₋₃alkylamino groups, particularly diC₁₋₄alkylamino groups such as methylamino, dimethylamino, ethylamino, diethylamino, propylamino, butylamino, and dibutylamino groups.

In the formula (I), R² may bond a carbon atom adjacent to a ring Z to form a ring. Such rings include a hydrocarbon ring or a heterocycle which is a 4- or more membered ring (for example, a 4- to 8-membered ring, preferably a 5- to 7-membered ring, more preferably a 5- to 6-membered ring). The hydrocarbon ring or the heterocycle may be an aromatic ring, however, usually a nonaromatic ring.

Exemplified as the hydrocarbon ring are 4- to 8-membered unsaturated aliphatic hydrocarbon rings such as cyclobutene ring, cyclopentene ring, cyclohexene ring, and cyclooctene ring (preferably, a 5- or 6-membered unsaturated aliphatic hydrocarbon rings), aromatic hydrocarbons such as benzene ring and naphthalene ring, and partially hydrogenated hydrocarbons thereof. Moreover, exemplified as the heterocycle is a heterocycle having at least one unsaturated bond among a heterocycle in the item of the ring Z hereinafter.

Exemplified as the the heterocycle having nitrogen atom as a hetero atom are a 5- or 6-membered monocyclic heterocycle such as pyrroline, pyrrole, imidazoline, and pyridine rings; and a condensed heterocycle in which a 5- or 6-membered heterocycle is condensed with a hydrocarbon ring, such as indoline ring. As the heterocycle having oxygen atom as a hetero atom, there are exemplified a 5- or 6-membered monocyclic heterocycle such as furan, pyran, oxacyclohexene, and 4H-1,3-dioxine rings, and a condensed heterocycle in which a 5- or 6-membered heterocycle is condensed with a hydrocarbon ring, such as chromane and isobenzofuran rings. Included among the examples of the heterocycle having sulfur atom as a hetero atom are a 5- or 6-membered monocyclic heterocycle such as 2H-thiophene ring, and a condensed heterocycle in which a 5- or 6-membered heterocycle is condensed with a hydrocarbon ring, such as thianthrene ring. Exemplified as heterocycles having different kinds of hetero atoms are a 5- or 6-membered monocyclic heterocycle such as oxazoline, and isoxazole rings; and a condensed heterocycle in which a 5- or 6-membered heterocycle is condensed with a hydrocarbon ring, such as phenoxathiin ring. Moreover, a heterocycle in which a part of the cycle is hydrogenated is also included. As a preferred cycle, there are exemplified the heterocycle having the oxygen atom as the hetero atom (particularly, the 5- or 6-membered monocyclic heterocycle). Incidentally, the heterocycle usually have a hetero atom at the position adjacent to a carbon atom bonding to a hydroxyl group in the compound of formula (I).

Although the hydrocarbon ring represented by the ring Z may be a non-aromatic hydrocarbon ring (e.g., C₃₋₂₀ cycloalkane rings such as cyclohexane and cyclooctane rings, C₃₋₂₀ cycloalkene rings such as cyclohexene ring), the ring Z is usually an aromatic hydrocarbon ring. It is sufficient that the aromatic hydrocarbon ring has at least a benzene ring, examples of which are benzene ring and condensed polycyclic aromatic hydrocarbon rings (e.g., naphthalene ring, anthracene ring, phenanthrene ring, phenalene ring). As the preferred hydrocarbon rings, there are exemplified benzene, naphthalene, and phenalene rings.

Included among the examples of the heterocycle represented by the ring Z are heterocycles having at least one hetero atom selected from nitrogen, oxygen, and sulfur atoms, and these may be condensed heterocycles in which a plurality of heterocycles are condensed together or condensed heterocycles in which a heterocycle is condensed (ortho-condensed, ortho and peri-condensed) with a hydrocarbon ring (non-aromatic hydrocarbon rings, or aromatic hydrocarbon rings), not limited to monocyclic heterocycles. Although it does not matter if the heterocycle represented by the ring Z is non-aromatic, it is usually aromatic.

Examples of the heterocycle having a nitrogen atom as a hetero atom are: a 5- or 6-membered monocyclic heterocycle such as pyrrole, imidazole, pyridine, and pyrazine rings; a condensed heterocycle in which a 5- or 6-membered heterocycle is condensed with a hydrocarbon ring such as indoline, quinoline, isoquinoline, quinazoline, carbazole, phenanthridine, acridine, and phenazine rings. As the heterocycle having an oxygen atom as a hetero atom, there are exemplified a 5- or 6-membered monocyclic heterocycle such as furan ring and a condensed heterocycle in which a 5- or 6-membered heterocycle is condensed with a hydrocarbon ring, such as isobenzofuran ring and chromene ring. Included among the examples of the heterocycle having a sulfur atom as a hetero atom are a 5- or 6-membered monocyclic heterocycle such as thiophene ring; and a condensed heterocycle in which a 5- or 6-membered heterocycle is condensed with a hydrocarbon ring, such as thianthrene ring. Exemplified as heterocycles having different kinds of hetero atoms are a 5- or 6-membered monocyclic heterocycle such as morpholine, isothiazole, and isoxazole rings; and a condensed heterocycle in which a 5- or 6-membered heterocycle is condensed with a hydrocarbon ring, such as phenoxathiin ring.

Preferred heterocycles include an aromatic heterocycle such 5- or 6-membered heterocycles having a nitrogen atom as a hetero atom (e.g., pyrrole ring, pyridine ring); and an aromatic heterocycle (e.g., carbazole ring) being an aromatic hydrocarbon ring (particularly, benzene ring or naphthalene ring) condensed with a 5- or 6-membered heterocycle having at least a nitrogen atom as a hetero atom.

When the ring Z is a heterocycle containing an aromatic ring, the ring Z is usually bonded to the adjacent C═C bond at the aromatic ring constituting the ring Z to form a conjugated system. Moreover, in the case of a polycyclic ring, insofar as the ring Z is bonded via its aromatic ring, it does not matter whether the other ring or rings are non-aromatic or aromatic ones, and part of the ring Z (and part of the non-conjugated system) may be hydrogenated. As the hydrocarbon ring partially hydrogenated, there are mentioned hydrogenated naphthalene rings such as 1,2-dihydronaphthalene ring, hydrogenated phenalene rings such as 2,3-dihydrophenalene and 2,3,3a,4,5,6-hexahydrophenalene rings. Moreover, as the heterocycle partially hydrogenated, there are mentioned julolidine ring and 9-formyljulolidine ring.

The ring Z may have a variety of substituents, examples of which are alkyl groups (e.g., C₁₋₆alkyl groups typified by methyl and ethyl groups); cycloalkyl groups (e.g., C₃₋₁₀cycloalkyl groups); aryl groups (e.g., C₆₋₁₈aryl groups typified by phenyl group); arylalkyl groups (e.g., C₆₋₁₂aryl-C₁₋₄alkyl groups typified by benzyl and diphenylmethyl groups); halogen atoms (fluorine, chlorine, bromine, and iodine atoms); hydroxyl group; alkoxy groups (e.g., C₁₋₆alkoxy groups such as methoxy and ethoxy groups); hydroxyalkyl groups (e.g., hydroxyC₁₋₆alkyl groups typified by hydroxymethyl group); carbonyl group; carboxyl group; alkoxycarbonyl groups (e.g., C₁₋₄alkoxycarbonyl groups); alkylcarbonyl groups (e.g., C₁₋₆alkyl-carbonyl groups); arylcarbonyl groups (e.g., C₆₋₁₂aryl-carbonyl groups); acyloxy groups (e.g., C₁₋₆acyloxy groups typified by acetyloxy group); cyano group; amino group; N-substituted amino groups (e.g., mono- or diC₁₋₆alkylamino groups typified by methylamino, dimethylamino, diethylamino, and methylethylamino groups, mono- or diC₆₋₁₈arylamino groups typified by phenylamino group, C₁₋₆acylamino groups typified by acetamide group); nitro group; and sulfonyl group.

Preferred substituents include C₁₋₄alkyl groups, C₆₋₁₂aryl groups, hydroxyl group, C₁₋₄alkoxy groups, amino group, mono- or diC₁₋₆alkylamino groups, mono- or diC₆₋₁₈arylamino groups, C₁₋₄acyloxy groups, C₁₋₄acylamino groups, etc. As the substituent(s), an electron donative group (e.g., amino group, N-substituted amino group) seems to be preferable.

There is no particular restriction as to the position(s) of the substituent(s) on the hydrocarbon ring or the heterocycle. For example, on the benzene ring, the substituent(s) is/are in the o-, m-, or p-position, and usually in the o- and/or p-position. Moreover, the hydrocarbon ring and the heterocycle each may have a plurality of substituents.

Exemplified as a hydrocarbon ring having such substituent(s) are benzene rings having a substituent(s) (e.g., benzene rings substituted with at least one substituent selected from hydroxyl group, a C₁₋₄alkoxy group, amino group, and a mono- or diC₁₋₄alkyl-substituted amino group). Moreover, exemplified as a heterocycle having a substituent(s) is a substituted (e.g., N-substituted) heterocycle in which its hetero atom(s) (e.g., nitrogen atom) is substituted with a C₁₋₆alkyl group [e.g., carbazole ring substituted with an N-C₁₋₄alkyl group].

In the formula (I) or (II), examples of combinations of substituents are exemplified below.

X¹: cyano group

X²: cyano group

R¹: a C₁₋₄alkyl or C₆₋₁₂aryl group

R²: an amino group or an N- mono- or diC₁₋₄alkylamino group, or a heterocycle formed together with a carbon atom adjacent to the ring Z (5- or 6-membered monocyclic heterocycle comprising an oxygen atom as a hetero atom) in the case of the formula (I)

Z: an aromatic ring having a substituent (at least one member selected from hydroxyl group, a C₁₋₄alkoxy group, amino group, and N-substituted amino group) [e.g., a benzene ring having the substituent; a condensed hydrocarbon ring having the substituent, in which a heterocycle is condensed with an aromatic hydrocarbon ring] or its ring in which non-conjugate part is partially hydrogenated.

Typical examples of the compound represented by the formula (I) are compounds in which the ring Z is a benzene ring which may have a substituent [e.g., 2,3-dicyano-5-amino-5-hydroxy-6-phenylmethylidene-7-methyl-4H-1,4-diazepine; 2,3-dicyano-5-amino-5-hydroxy-6-(4-C₁₋₄alkoxyphenylmethylidene)-7-methyl-4H-1,4-diazepines such as 2,3-dicyano-5-amino-5-hydroxy-6-(4-methoxyphenylmethylidene)-7-methyl-4H-1,4-diazepine; 2,3-dicyano-5-amino-5-hydroxy-6-(4-mono or diC₁₋₄alkylaminophenylmethylidene)-7-methyl-4H-1,4-diazepines such as 2,3-dicyano-5-amino-5-hydroxy-6-(4-dimethylaminophenylmethylidene)-7-methyl-4H-1,4-diazepine; those with amino group in the 5-position being an N- mono- or diC₁₋₄alkylamino group]; compounds in which the ring Z is a condensed polycyclic hydrocarbon ring or the ring Z is a condensed heterocycle of a heterocycle condensed with an aromatic hydrocarbon ring in the above compound, or its partially hydrogenated ring [e.g., 2,3-dicyano-5-mono- or diC₁₋₄alkylamino-5-hydroxy-6-(naphthalen-2-yl-methylidene)-7-methyl-4H-1,4-diazepine, 2,3-dicyano-5-mono- or diC₁₋₄alkylamino-5-hydroxy-6-(9-ethyl-3-carbazolylmethylidene)-7-methyl-4H-1,4-diazepine)].

Moreover, exemplified as the compound represented by the formula (I) are, in the above exemplified compound, a compound in which R² bonds to a carbon atom adjacent to a ring Z to form a ring [e.g., 2,3-dicyano-4a-hydroxy-9-methyl-8-(4-mono- or diC₁₋₄alkylaminophenyl)-4H,6H,7H-oxacycloC₄₋₈alkano[2,3-e]-1,4-diazepine], compounds in which the ring Z is a condensed polycyclic hydrocarbon ring or the ring Z is a condensed heterocycle in which a heterocycle is condensed with an aromatic hydrocarbon ring in the above compound, or its partially hydrogenated ring.

Exemplified as the typical compound represented by the formula (II) are compounds in which the ring Z is a benzene ring which may have a substituent [e.g., 2,3-dicyano-5-amino-7-(2-phenylethene-1-yl)-6H-1,4-diazepine; 2,3-dicyano-5-amino-7-(2-(4-C₁₋₄alkoxyphenyl)ethene-1-yl)-6H-1,4-diazepines such as 2,3-dicyano-5-amino-7-(2-(4-methoxyphenyl)ethene-1-yl)-6H-1,4-diazepine; 2,3-dicyano-5-amino-7-(2-(4-mono- or diC₁₋₄alkylaminophenyl)ethene-1-yl)-6H-1,4-diazepines such as 2,3-dicyano-5-amino-7-(2-(4-dimethylaminophenyl)ethene-1-yl)-6H-1,4-diazepine; those with the amino group in the 5-position being an N-mono- or diC₁₋₄alkylamino group]; compounds in which the ring Z is a condensed polycyclic hydrocarbon ring or the ring Z is an aromatic heterocycle of a heterocycle condensed with an aromatic hydrocarbon ring in the above compound, or its partially hydrogenated ring [e.g., 2,3-dicyano-5-mono- or diC₁₋₄alkylamino-7-(2-(phenalen-2-yl)ethene-1-yl)-6H-1,4-diazepine, 2,3-dicyano-5-mono- or diC₁₋₄alkylamino-7-[(9-ethyl-3-carbazolyl)vinyl-1-yl]-6H-1,4-diazepine)].

### Production Process

The compound of the present invention can be prepared, for example, in accordance with the following reaction formula (1).

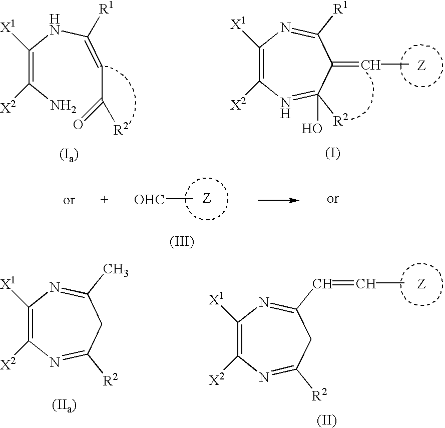

wherein X¹, X², R¹, R², and the ring Z have the same meanings as defined above.

The compound of the formula (I) or (II) can be obtained by reacting the compound represented by the formula (Iₐ) or (IIₐ) (including its structural isomers) with the compound represented by the formula (III).

As compounds represented by formula (Ia), there are exemplified N-substituted diaminomaleonitriles [e.g., 1,2-dicyanoethenes such as 1-[N-(4-C₁₋₄alkylamino-4-oxo-2-butenyl)]amino-2-amino-1,2-dicyanoethene]. As compounds represented by formula (IIa), there are exemplified 2,3-dicyano-diazepines [e.g., azepines corresponding to the compound of the formula (II) such as 2,3-dicyano-5-amino or N-C₁₋₄alkylamino-7-methyl-1,4-diazepines.

Typical examples of the compound represented by the formula (III) are aldehydes [e.g., aldehydes in which the ring z is a benzene ring (e.g., benzaldehyde, halobenzaldehyde, aminobenzaldehyde, N-substituted aminobenzaldehyde (particularly, N-C₁₋₄alkyl substituted aminobenzaldehyde), phenol-aldehyde, C₁₋₄alkoxybenzaldehyde); aldehydes in which the ring Z is a condensed polycyclic hydrocarbon ring (e.g., naphthalenecarbaldehyde, phenalenecarbaldehyde); aldehydes in which the ring Z is a 5- or 6-membered heterocycle containing nitrogen atom as a hetero atom, or a condensed heterocycle of a heterocycle and a hydrocarbon ring (e.g., 9-ethyl-3-formylcarbazole)]. The amount of the compound represented by the formula (III) is, per 1 mol of the compound of the formula (Iₐ) or (IIₐ), about 1 to 3 mol, preferably about 1 to 1.5 mol.

The reaction described above can be carried out in the presence of a solvent inert to the reaction, such as an aliphatic hydrocarbon (e.g., hexane), an alicyclic hydrocarbon (e.g., cyclohexane), an aromatic hydrocarbon (e.g., benzene, toluene), a halogenated hydrocarbon (e.g., chloroform), an alcohol (e.g., methanol, ethanol, isopropyl alcohol, butanol), an ester (e.g., ethyl acetate, butyl acetate, isobutyl acetate), an ether (e.g., dioxane, diethyl ether, teterahydrofuran), an amide (e.g., formamide, acetamide, dimethylformamide (DMF), dimethylacetamide), a nitrile (e.g., acetonitrile, benzonitrile), a sulfoxide (e.g., dimethylsulfoxide). If necessary, a catalyst (e.g., a basic catalyst such as pyridine and piperidine) may be used. The amount of the catalyst to be used can be selected within the range of, per 1 mol of the compound of the formula (Iₐ) or (IIₐ), about 0.001 to 1 mol.

When a solvent is used, the reaction temperature can be selected within the range of about 0° C. to reflux temperature and is for example about 50 to 120° C., preferably about 60 to 100° C. The reaction can be effected under ordinary pressure, reduced pressure, or applied pressure. The reaction may be carried out in an atmosphere of an inert gas (e.g., nitrogen, argon, helium).

After the completion of the reaction, the compound (I) or (II) formed in the above-described reaction can easily be separated and purified by such a conventional means as filtration, condensation, distillation, extraction, crystallization, recrystallization, column chromatography, or a combination means thereof.

In the above reaction, a compound of the formula (Iₐ) or (IIₐ) reacts with aldehydes efficiently to form a C═C double bond, probably because the structual isomers of compounds of the formulae (Iₐ) and (IIₐ) have an enamine skeleton. This is why, a reaction of a compound represented by the formula (Iₐ) or (IIₐ) and aldehydes (III) seems to occurred on a part (or position) having the enamine skeleton or a part to easily form the enamine skeleton (that is, a part capable of forming a conjugated system (a part capable of forming a planar structure)). That is, in the compound of the formula (Iₐ), since a carbon atom in β- or γ-position to R² is planer and capable of forming a conjugated system, any one of the above carbon atoms is easy to form the enamine skeleton. Therefore, a part reacted with the aldehydes is any one of the above carbon atoms.

Meanwhile, in the reaction the compound of the formula (IIₐ) and aldehydes (III), since carbon atom in 6-position of the compound of the formula (IIₐ) is not planar, the reaction is unable to form an enamine skeleton. As a result, the reaction of a carbon atom in 7-position capable of forming the enamine skeleton and aldehydes seems to occur.

Incidentally, in the formula (Iₐ), when R² forms a ring together with a carbon atom in β-position, a carbon atom in 6-(β-)position reacts with aldehydes to occur ring-expansion, despite the carbon atom in 6-(β-)position forms a ring. The obtained compound is a compound in which the number of ring members increases by one.

Incidentally, as the compound of the formula (Iₐ) or (IIₐ) and the aldehydes (III), commercial products may be used, and they may be produced by conventional manners. Particularly, the compound represented by the formula (Iₐ) or (IIₐ) may be prepared by the following reaction (2).

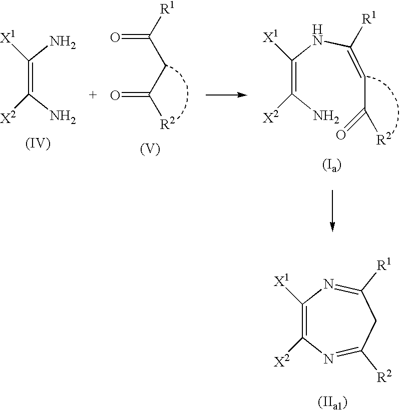

wherein X¹, X², R¹, and R² have the same meanings as defined above.

The compound of the formula (Iₐ) can be formed by reacting (condensing) the compound represented by the formula (IV) with the compound represented by the formula (V). The compound of the formula (IIₐ₁) can be obtained by, if necessary, dehydrating the compound of the formula (Iₐ). The compound of the formula (IIₐ) corresponds to the compound of the formula (IIₐ₁) with methyl group as R¹.

Typical examples of the compound represented by the formula (IV) are diamines [e.g., 1,2-dicyano-1,2-diaminoethene (diaminomaleonitrile), 1-cyano-2-(dimethylamino)-1,2-diaminoethene, 1,2-dicyano-2-(benzylamino)-1-aminoethene)]. Typical examples of the compound represented by the formula (V) are diketone compounds [e.g., 1,3-diC₂₋₁₂acylmethanes such as acetylacetone, benzoylacetone, and dibenzoylmethane; 3-C₂₋₁₂acyl-cycloalkan-2-ones such as 3-acetylcyclopenta-2-one, 1-oxa-3-acetylcyclopenta-2-one, 1-oxa-3-acetylcyclohexa-2-one, 3-benzylcyclohexa-2-one, and 1-oxa-3-benzylcyclopenta-2-one. Incidentally, such diamines and diketones may be commercial products or may produce by conventional manners. For example, diketones in which R² forms a ring with a carbon atom in β-position can be produced by subjecting diketones having a reactive group (e.g., a hydroxyl group, a carboxyl group, an amino group, a halogen atom) in both carbon atom of R² and carbon atom in p-position to condensation reaction (e.g., a condensation reaction of hydroxyl group and carboxyl group, a condensation reaction of carboxyl group and amino group).

The amount of the compound of the formula (V) to be used is usually about 1 to 3 mol, preferably about 1 to 1.5 mol relative to 1 mol of the compound of the formula (IV).

The condensation reaction described above may be effected in the presence or absence of a catalyst. Exemplified as the catalyst are conventional ones, such as acid catalysts (e.g., inorganic acids such as sulfuric acid, phosphoric acid, hydrochloric acid; organic acids such as acetic acid, sulfonic acid, p-toluenesulfonic acid) and basic catalysts (e.g., amines such as piperidine, hydroxides or oxides of alkali metals or alkaline earth metals). The amount of the catalyst to be used can be selected within the range of about 0.001 to 1 mol relative to 1 mol of the compound of the formula (IV). A dehydrating agent (e.g., phosphorus pentoxide) may additionally be used.

The condensation reaction may be effected in a solvent inert to the reaction. As the solvent, those listed above are available (e.g., benzene). When the solvent is used, the reaction temperature can be selected within the range of about 0° C. to reflux temperature, and is for example about 50 to 120° C., preferably about 60 to 100° C. It is possible to effect the reaction under ordinary, reduced, or applied pressure. The reaction may be effected in an atmosphere of an inert gas (e.g., nitrogen, argon, helium). After the completion of the reaction, the compound formed by the condensation reaction described above can be easily separated and purified by any separating means of those mentioned above.

The compound of the present invention is characterized in that, due to its specific structure represented by the formula (I) or (II) (that is, an azepine ring and a ring Z), it is capable of emitting light by being supplied with energy externally (irradiation of light, the action of an electric field). There is no particular restriction as to the irradiation of light so far as light is of a certain wavelength capable of exciting the azepine compound of the formula (I) or (II). For example, ultraviolet rays (400 nm or less) and visible rays [about 360 to 860 nm (preferably about 400 to 760 nm, more preferably about 400 to 700 nm)] can be used. The emission wavelength varies over a wide range (e.g., about 400 to 900 nm, preferably about 400 to 800 nm, more preferably about 400 to 700 nm (violet to red)), depending on, for example, the kind(s) of the substituent(s) and the position of substitution. The azepine compound (I) or (II) of the present invention expresses, though varies for different kinds of substituents or positions of substitution, violet to red color (particularly, yellow to red) and has a large molar absorption coefficient. This color (developing) system of the compound may be because the intramolecular charge transfer in which the azepine ring having the cyano group etc within its molecule act as a strong electron attractive group (acceptor) and the aromatic ring being the ring Z act as an electron donative group.

Upon irradiation of light (particularly, visible rays), the compound of the formula (I) or (II) emits fluorescent light in a solution. The wavelength of fluorescence varies within the range mentioned above. Particularly, although the azepine ring of the compound of the present invention has a non-planar structure, generally, it seems to have a tendency to emit fluorescent light of relatively long wavelengths (about 500 to 700 nm, preferably about 530 to 700 nm: yellow to red), probably because the azepine ring acts as a strong electron attractive group.

Under the action of an electric field (injection of a carrier), the compound of the present invention is capable of emitting light. The emission wavelength can be selected within the range mentioned above. Moreover, the compound of the present invention is capable of emitting light of relatively long wavelengths (about 500 to 700 nm, preferably about 550 to 700 nm: yellow to red). Therefore, the compound of the present invention is useful as an emission center compound for an organic EL device.

Incidentally, in the formulae (I) and (II), the substituent effects on an absorption coefficient and a luminescence wavelength can be confirmed by comparing absorption spectrums and fluorescence spectrums of the compounds varying in substituent.

For example, in the absorption spectrum, the absorption coefficient of the formula (I) or (II) is varied with the substituent of R². For example, in the case where R² is a group having a small degree of freedom of rotation (e.g., mono- or diC₁₋₄alkylamino group) or a bulky group (e.g., C₆₋₁₂aryl group), the absorption coefficient become small due to a steric hindrance. Moreover, in a fluorescence spectrum, in the case where R² is a group which easily forms a conjugated system (e.g., an aromatic group such as C₆₋₁₂aryl group), a maximum of absorption is positioned in the region of long wavelength. Therefore, in the compound of the formula (II), since there are three double bonds in an azepine ring, it is easy to form the π-conjugated system and emit fluorescence in the region of long wavelength compared with the compound of the formula (I) having a similar R² group. As described above, even though compounds have a similar skeleton, the emission wavelength is controllable over a wide range and capable of emitting light of longer wavelength by choosing a substituent. Moreover, the emission wavelength is also controllable corresponding to an ease of energy transfer (ease of forming a conjugated system).

In case of that R² group is an electron-donating group (e.g., diC₁₋₄alkylamino groups), a fluorescence is sometimes emitted in the range of wavelength different from that in case of an intramolecular charge transfer between a ring Z and a azepine ring, since different intramolecular charge transfer from R² group to an azepine ring occurs. In this case, the respective fluorescence intensity is smaller than that of a compound having no electron-donating group as R² group.

Moreover, the compound of the formula (I) or (II) contained in a solid (e.g., a thin film on which the compound is vapor-deposited) shows the same properties (emission of light upon irradiation of light or by the action of an electric field) as those shown in the case of the compound being in a solution. Therefore, the compound of the present invention can be also used in the form of a solid (e.g., as a film, powder, particles), and its use is not restricted. Incidentally, in a solid, a fluorescence is sometimes emitted longer wavelength than a wavelength of fluorescence in a liquid owing to a intermolecular interaction.

So that the compound of the present invention is capable of emitting light upon irradiation of light or by the action of an electric field, it can be utilized in various fields as a functional material. For example, the compound of the present invention is useful not only as a fluorescent material (e.g., a fluorescent pigment, a fluorescent flaw detecting agent, a fluorescent white dye, particularly as a fluorescent material such as a fluorescent dye) but also as a material for display (e.g., light emitting device material such as an electroluminescence material).

### Organic Electroluminescence Device

The electroluminescence (EL) device of the present invention is composed of a pair of electrodes and an organic layer interposed therebetween. The organic layer comprises at least the compound represented by the aforementioned formula (I) or (II). Particularly, the layer containing the compound of the formula (I) or (II) forms a light-emitting region, constituting a light-emitting layer. The light-emitting layer may be formed of a film-formable compound of the formula (I) or (II) singly, or may be formed of a film-formable or non-film-formable compound of the formula (I) or (II) and a binder having a film-forming property. As the binder, a resin having a film-forming property (a thermoplastic resin, a thermosetting resin) can be usually used.

Examples of the thermoplastic resin are olefinic resins such as polyethylene, polypropylene, ethylene-propylene copolymer, and polybutene; styrenic resins such as polystyrene, rubber-modified polystyrene (e.g., HIPS), acrylonitrile-styrene copolymer, and acrylonitrile-butadiene-styrene copolymer; acrylic resins [e.g., homo- or copolymers of (meth)acrylic monomers (e.g., C₁₋₆alkyl (meth)acrylates such as methyl (meth)acrylate, ethyl (meth)acrylate, and butyl (meth)acrylate; hydroxyC₂₋₄alkyl (meth)acrylates such as hydroxyethyl (meth) acrylate and hydroxypropyl (meth) acrylate; glycidyl (meth)acrylate; (meth)acrylic acid; (meth)acrylonitrile); copolymers of the (meth)acrylic monomers mentioned above with copolymerizable monomers (e.g., aromatic vinyl monomers such as styrene) (e.g., methyl methacrylate-styrene copolymer)]; vinyl-series resins such as vinyl alcohol-series polymers typified by polyvinyl alcohol and ethylene-vinyl alcohol copolymer, polyvinyl chloride, vinyl chloride-vinyl acetate copolymer, polyvinylidene chloride, polyvinyl acetate, ethylene-vinyl acetate copolymer, and polyvinylacetyl; polyamide-series resins such as 6-nylon, 6,6-nylon, 6,10-nylon, and 6,12-nylon; polyester resins [e.g., polyalkylene terephthalates (e.g., polyethylene terephthalate, polybutylene terephthalate), alkylene arylate-series resins such as polyalkylene naphthalate, alkylene arylate copolyester resins]; fluorine-containing resins; polycarbonate; polyacetal; polyphenylene ether; polyphenylene sulfide; polyether sulfone; polyether ketone; thermoplastic polyimide; thermoplastic polyurethane; and norbornene-series polymer.

Exemplified as the thermosetting resin are phenolic resins, amino resins (e.g., urea resins, melamine resins), thermosetting acrylic resins, unsaturated polyester resins, alkyd resins, diallyl phthalate resins, epoxy resins, and silicone resins.

These binders can be used either singly or in combination.

The content of the compound of the formula (I) or (II) is, per 100 parts by weight of the binder, about 0.01 to 10 parts by weight, preferably about 0.05 to 5 parts by weight, more preferably about 0.1 to 3 parts by weight.

If necessary, into the light-emitting layer may be incorporated other emission center compounds (or luminescent compounds), examples of which are heterocyclic compounds having at least one hetero atom selected from oxygen, nitrogen, and sulfur atoms [e.g., bis(C₁₋₆alkyl-benzoxazoyl)thiophene typified by 2,5-bis(5-tert-butyl-2-benzoxazoyl)-thiophene; nile red; coumarins typified by coumarin 6 and coumarin 7; 4-(dicyanoC₁₋₄alkylene)-2-C₁₋₄alkyl-6-(p-diC₁₋₄alkylaminostyryl)-4H-pyran typified by 4-(dicyanomethylene)-2-methyl-6-(p-dimethylaminostyryl)-4H-pyran; and quinacridone]; condensed polycyclic hydrocarbons such as rubrene and perylene; tetraC₆₋₁₂aryl-1,3-butadiene such as 1,1,4,4-tetraphenyl-1,3-butadiene (TPB); bis(2-(4-C₁₋₄alkylphenyl)C₂₋₄alkynyl)benzene such as 1,4-bis(2-(4-ethylphenyl)ethynyl)benzene; and bis(2,2′-diC₆₋₁₂arylvinyl)biphenyl such as 4,4′-bis(2,2′-diphenylvinyl)biphenyl. These emission center compounds can be used either singly or in combination.

The content of the emission center compound is selected within a range not adversely affecting the efficiency of emission of the compound of the formula (I) or (II) and about 0.01 to 10 parts by weight, about 0.05 to 5 parts by weight, more preferably about 0.1 to 3 parts by weight relative to 100 parts by weight of the binder. The proportion of the compound of the formula (I) or (II) to the other emission center compound(s) is former/latter (weight ratio)=about 40/60 to 100/0, preferably about 50/50 to 95/5, more preferably about 60/40 to 90/10.

If necessary, the light-emitting layer comprising the compound of the formula (I) or (II) may be given an electron-transporting function and/or a hole-transporting function. For the purpose of giving such function(s), (1) to the light-emitting layer may be added organic polymers or compounds having the functions described above; or (2) the light-emitting layer may be laminated with a layer or layers having the functions described above. In the case (1), it is possible to fabricate an organic EL device having a single-layered structure.

Exemplified as the organic polymer having at least one function selected from the electron-transporting and hole-transporting functions are vinyl-series polymers having at least one functional group selected from hole-transporting functional groups and electron-transporting functional groups in the main chain or side chain, such as polyphenylenevinylenes [e.g., homo- or copolymers of C₆₋₁₂arylenevinylenes which may have a substituent (e.g., C₁₋₁₀alkoxy groups), such as polyphenylenevinylene, poly(2,5-dimethoxyphenylenevinylene, and polynaphthalenevinylene]; polyphenylenes (particularly, polyparaphenylenes) [e.g., homo- or copolymers of phenylenes which may have a substituent (e.g., C₁₋₁₀ alkoxy groups), such as polyparaphenylene and poly-2,5-dimethoxyparaphenylene]; polythiophenes [e.g., polyC₁₋₂₀alkylthiophenes such as poly(3-alkylthiophene); polyC₃₋₂₀cycloalkylthiophenes such as poly(3-cyclohexylthiophene); homo- or copolymers of C₆₋₂₀arylthiophenes which may have a substituent (e.g., C₁₋₁₀alkyl groups) such as poly(3-(4-n-hexylphenyl)thiophene)]; polyfluorenes such as polyC₁₋₂₀alkylfluorenes; vinyl-series polymers having at least one functional group selected from a hole-transporting functional group and an electron-transporting functional group in the main or side chain, such as poly-N-vinylcarbazole (PVK), poly-4-N,N-diphenylaminostyrene, poly(N-(p-diphenylamino)phenylmethacrylamide), poly(N,N′-diphenyl-N,N′-bis(3-methylphenyl)-1,1′-biphenyl-4,4′-diaminomethacrylamide) (PTPDMA), and poly-4-(5-naphthyl-1,3,4-oxadiazole)styrene; polyC₁₋₄alkylphenylsilanes such as polymethylphenylsilane; polymers derived from an aromatic amine derivative; and copolymers thereof. These resins can be used either singly or in combination. As preferred resins, there are exemplified are polyN-vinylcarbazole or copolymers of which the main component (50% or more by weight, preferably 60 to 98% by weight) is N-vinylcarbazole and polymers derived from an aromatic amine derivative (polymers having the functional group in the side or main chain derived from an aromatic amine derivative) in the main or side chain.

PVK is amorphous and excellent in heat resistance (glass transition temperature Tg: 224° C.). Although there is no particular restriction on the degree of polymerization of PVK, it is for example about 100 to 1,000, preferably about 200 to 800.

In the case where the light-emitting layer is comprised of the compound of the formula (I) or (II) and the organic polymer described above, the content of the compound of the formula (I) or (II) is, per 100 parts by weight of the organic polymer, about 0.01 to 10 parts by weight, preferably about 0.05 to 5 parts by weight, more preferably about 0.1 to 3 parts by weight.

If necessary, to the light-emitting layer comprised of the compound of the formula (I) or (II) and the organic polymer may be added a compound having an electron- or hole-transporting function.

Examples of the compound having an electron-transporting function are oxadiazole derivatives [e.g., oxadiazole derivatives having a C₆₋₂₀aryl group which may have a substituent, such as 2-(4-biphenyl)-5-(4-tert-butylphenyl)-1,3,4-oxadiazole (PBD), 2,5-bis(1-naphtyl)-1,3,4-oxadiazole (BND), 1,3-bis[5-(4-tert-butylphenyl)-1,3,4,-oxadiazole)]benzene (BPOB), 1,3,5-tris[5-(4-tert-butylphenyl)-1,3,4-oxadiazole]benzene (TPOB), and 1,3,5-tris[5-(1-naphtyl)-1,3,4-oxadiazole]benzene (TNOB); diphenoquinones [e.g., diphenoquinones which may have a substituent (e.g., C₁₋₁₀ alkyl groups), such as 3,5,3′,5′-tetrakis-tert-butyldiphenoquione; 1,2,3,4,5-pentaphenyl-1,3-cyclopentadiene (PPCP); and quinolinic acid complexes such as tris(8-quinolinorato)aluminium (III) complex, bis(benzoquinolinorato)beryllium complex, and tris(10-hydroxybenzo[h]quinolilate)beryllium complex, with oxadiazole derivatives (e.g., PBD) particularly preferred.

As the compound having a hole-transporting function, there may be exemplified aromatic tertiary amines such as N,N′-diphenyl-N,N′-bis(3-methylphenyl)-1,1′-biphenyl-4,4′-diamine (TPD), N,N′-diphenyl-N,N′-bis(1-naphthyl)-1,1′-biphenyl-4,4′-diamine (NPD), 1,1-bis[(di-4-tolylamino)phenyl]cyclohexane, N,N,N′N′-tetra(3-methylphenyl)-1,3-diaminobenzene (PDA), 4,4′,4″-tris(3-methylphenylphenylamino)triphenylamine (m-MTDATA), 4,4′,4″-tris(1-naphthylphenylamino)triphenylamine(1-TNATA), 4,4′,4″-tris(2-naphthylphenylamino)triphenylamine (2-TNATA), 4,4′,4″-tri(N-carbazolyl)triphenylamine (TCTA), 1,3,5-tris[4-(3-methylphenylphenylamino)phenyl]benzene (m-MTDAPB), and triphenylamine; and phthalocyanines.

The compounds having en electron-transporting function and those having a hole-transporting function may be used either singly or in combination.

The content of the compound having en electron and/or hole-transporting function is, relative to 100 parts by weight of the binder (and/or the organic polymer described above), about 10 to 200 parts by weight, preferably about 30 to 150 parts by weight, more preferably about 50 to 130 parts by weight.

When the light-emitting layer is lacking in either the electron-transporting function or the hole-transporting function, or attempting to improve each function, a layer or layers having the desired function may be applied onto the light-emitting layer by a conventional method (e.g., vapor deposition, solution coating). These layers may be of low molecular weight compounds or high molecular weight compounds.

The thickness of each layer constituting the organic layer is not particularly limited, and is for example about 1 nm to 1 μm, preferably about 5 to 800 nm, more preferably about 10 to 500 nm, particularly about 15 to 300 nm.

As the anode of the organic EL device, for example, a transparent electrode (e.g., indium-tin-oxide (ITO) electrode) formed by a conventional process (e.g., vacuum deposition) is employed. As the cathode, a highly conductive metal of low work function (e.g., magnesium, lithium, aluminum, silver) is used. In the case where magnesium is employed as the cathode, for improving the adhesion to a film for organic EL devices, magnesium may be deposited together with a small amount of silver (e.g., 1 to 10% by weight).

There is no particular restrictions on the process for the production of the organic electroluminescence device of the present invention, and conventional ones are adoptable. For example, the organic layer (e.g., light-emitting layer) is formed by forming a coat being the aforementioned transparent electrode (e.g., ITO electrode) on a transparent substrate and then applying or casting a coating solution comprising the compound of the formula (I) or (II) in a conventional manner (e.g., spin coating, casting). The organic electroluminescence device is fabricated by further forming a cathode on the organic layer by vapor deposition or other means. If necessary, the anode or the light-emitting layer may be laminated with a layer or layers having an electron- and/or hole-transporting function by such a conventional method as vapor deposition or coating.

Examples of the substrate are those transparent enough to transmit light emitted by the emission center compound (e.g., glass plates such as soda glass, non-alkali glass, and quartz glass, sheets or films of polymers such as polyester, polysulfone, and polyethersulfone). When fabricating a flexible organic EL device, the use of a polymer film is preferred.

Although the thickness of the organic EL device (e.g., the organic layer and the electrodes) as a whole is not particularly limited, it is about 50 nm to 10 μm, preferably about 100 nm to 8 μm, more preferably about 300 nm to 5 μm.

According to the present invention, since an azepine compound having a specific structure is employed as the organic layer (particularly, light-emitting layer) of the organic EL device, it is possible to control the wavelength of light the organic EL device emits. Moreover, according to the present invention, in spite of the fact that an azepine ring of the azepine compound has a non-planar structure, the compound can emit light of relatively long wavelength (e.g., about 530 to 700 nm, yellow to red) and provides an organic EL device of high luminance and high durability.

The compound of the present invention can emit light by being irradiated with light or by the action of an electric field because it has an azepine ring and a specific ring Z. Therefore, the compound of the present invention is useful as a functional material typified by a fluorescent material and a material for display devices. In particular, a compound which emits light by the action of an electric field is particularly useful as an emission center compound for use in an organic electroluminescence device and is capable of emitting light within a wide wavelength range. Therefore, it is possible to control the wavelength of light to be emitted by an organic EL device.

### EXAMPLES

The following examples are intended to describe this invention in further detail and should by no means be interpreted as defining the scope of the invention.

Analysis manners of examples are described as follows.

¹H-NMR spectrum and ¹³C-NMR spectrum were measured in deuterium chloroform or deuterium dimethylsulfoxide with the use of tetrachloromethylsilane as an internal standard by Varian Unity-plus 300 NMR spectroscope. Mass spectrum was measured by GCMS-QP5000 spectroscope (manufactured by Shimadzu Seisakusho K.K.). Melting point was measured by the melting point measuring equipment MP-21 (manufactured by Yamamoto) without correcting. Elemental analysis is measured by CHN MT-3 recorder (manufactured by Yanaco). Wako gel C-300 (silica gel) was used for column chromatography.

According to UV/visible spectrum and fluorescence spectrum, azepine compounds obtained in examples and synthesis examples were dissolved in chloroform respectively to be 1.5×10⁻⁵ mol/l, and the azepine compounds were measured by U-2010 spectroscope (manufactured by Hitachi K.K.) and F-4500 fluorescence spectroscope (manufactured by Hitachi K.K.).

As a starting material, diaminomaleonitrile manufactured by Nihon Soda K.K., 1-dimethylaminobutane-1,3-dione and 3-acetyloxacyclopenta-2-one manufactured by Daicel Kagaku K.K. were used. Commercial products were used for other reagents except above reagents, which were without purifying.

### Synthesis Example 1

Into a flask provided with Dean-Stark trap were fed 50 ml of benzene, 10 mmol of diaminomaleonitrile, 10 mmol of 1-phenylbutane-1,3-dione as diketones and 30 mg of oxalic acid, and the mixture was subjected to reflux for 3 hrs and the resulting water was removed. The mixture was cooled to room temperature, and then benzene was removed under reduced pressure. The residue was washed with water and filtered. The precipitate was isolated by column chromatography on silica gel with the use of chloroform as an eluate, and purified by recrystallization to give N-(4-phenyl-4-oxo-2-buten-2-yl)diaminomaleonitrile (3b) in 82% yield. The results of analyses are described as follows.

melting point (decomposition): 146 to 148° C.

NMR δ_{H}(CDCl₃): 12.26 (1H, broad, NH), 7.83 (2H, d, phenyl protons), 7.47 (3H, m, phenyl protons), 5.95 (1H, s, 6-H), 5.23 (2H, broad, NH₂), 2.07 (3H, s, CH₃)

| Elemental analysis | C (%) | H (%) | N (%) |
|---|---|---|---|
| Calculated | 66.65 | 4.79 | 22.21 |
| Found | 65.94 | 4.74 | 23.13 |
| mass spectrum m/e(M⁺): | 252 | | |

### Synthesis Example 2

The object compound N-(4-ethoxy-4-oxo-2-buten-2-yl)diaminomaleonitrile (3c) in 58% yield was obtained in the same manner as in Synthesis example 1 with the exception that 1-ethoxybutane-1,3-dione was used in lieu of 1-phenylbutane-1,3-dione as diketones. The results of analyses are described as follows.

melting point (decomposition): 154 to 155° C.

NMR δ_{H}(CDCl₃): 9.45 (1H, s, NH), 4.91 (1H, broad, 6-H), 4.68 (2H, broad, NH₂), 4.13 (2H, q, J=7.2, CH₂), 2.06 (3H, s, CH₃), 1.28 (3H, t, J=7.2, CH₃)

| Elemental analysis | C(%) | H(%) | N(%) |
|---|---|---|---|
| Calculated | 54.54 | 5.49 | 25.44 |
| Found | 54.33 | 5.57 | 25.46 |

mass spectrum m/e(M⁺): 220

### Synthesis Example 3

The object compound N-(4-dimethylamino-4-oxo-2-buten-2-yl)diaminomaleonitrile (3d) in 83% yield was obtained in the same manner as in Synthesis example 1 with the exception that 1-dimethylaminobutane-1,3-dione was used in lieu of 1-phenylbutane-1,3-dione as diketones. The results of analyses are described as follows.

melting point (decomposition): 149 to 151° C.

NMR δ_{H}(d₆-DMSO): 10.34 (1H, s, NH), 7.40 (2H, s, NH₂), 5.17 (1H, s, 6-H), 2.96 (3H, broad, NMe), 2.83 (3H, broad, NMe), 1.89 (3H, s, CH₃); (CDCl₃): 3.55 (2H, s, NH₂), 3.00 (3H, s, NMe), 2.93 (3H, s, NMe), 2.29 (3H, s, CH₃),

| Elemental analysis | C (%) | H (%) | N (%) |
|---|---|---|---|
| Calculated | 54.78 | 5.98 | 31.94 |
| Found | 54.66 | 5.96 | 31.92 |
| mass spectrum m/e(M⁺): | 219 | | |

### Synthesis Example 4

The object compound N-[1-(2-oxooxacyclopentan-3-ylidene)ethyl]diaminomaleonitrile (3e) in 75% yield was obtained in the same manner as in Synthesis example 1 with the exception that 3-acetyloxacyclopenta-2-one was used in lieu of 1-phenylbutane-1,3-dione as diketones. The results of analyses are described as follows.

melting point (decomposition): 147 to 148° C.

NMR δ_{H}(CDCl₃): 8.97 (1H, s, NH), 4.79 (2H, broad, NH₂), 4.39 (2H, t, J=7.8, OCH₂), 2.91 (2H, t, J=7.8, CH₂), 2.08 (3H, d, J=0.9, CH₃); (d₆-DMSO): 8.50 (1H, s, NH), 7.59 (2H, s, NH₂), 4.26 (2H, t, J=7.8, OCH₂), 2.83 (2H, t, J=7.8, CH₂), 1.90 (3H, s, CH₃),

| Elemental analysis | C (%) | H (%) | N (%) |
|---|---|---|---|
| Calculated | 55.04 | 4.62 | 25.68 |
| Found | 54.80 | 4.64 | 25.69 |
| mass spectrum m/e(M⁺): | 218 | | |

### Synthesis Example 5

Into a flask were fed 200 ml of ethanol, 50 mmol of diaminomaleonitrile, 50 mmol of 1-methylbutane-1,3-dione as diketones and 2.6 g of phosphorus pentaoxide, and the mixture was subjected to reflux for 6 hrs. The mixture was cooled to room temperature, and then ethanol was evaporated to be 40 ml of ethanol. The precipitate was filtered, dried, and then isolated by column chromatography on silica gel with the use of chloroform as an eluate, and purified by recrystallization to give 2,3-dicyano-5,7-dimethyl-6H-1,4-diazepine (4a). The results of analyses are described as follows.

melting point (decomposition): 199 to 200° C.

NMR δ_{H}(CDCl₃): 4.27 (1H, broad, CH₂), 2.30 (6H, s, 2CH₃), 1.85 (1H, broad, CH₂)

mass spectrum m/e(M⁺): 172

### Synthesis Example 6

The object compound 2,3-dicyano-7-methyl-5-phenyl-6H-1,4-diazepine (4b) was obtained in the same manner as in Synthesis example 5 with the exception that 1-phenylbutane-1,3-dione was used in lieu of 1-methylbutane-1,3-dione as diketones. The results of analyses are described as follows.

melting point (decomposition): 126 to 127° C.

NMR δ_{H}(CDCl₃): 8.01 (2H, d, J=8.2, phenylprotons), 7.62 (1H, m, phenyl proton), 7.56 (2H, m, phenyl protons), 5.06 (1H, d, J=10, CH₂), 2.22 (3H, s, CH₃), 1.87 (1H, d, J=10, CH₂)

mass spectrum m/e(M⁺): 234

### Synthesis Example 7

Into a flask provided with Dean-Stark trap were fed 50 ml of benzene, 5 mmol of the compound 3b of Synthesis example 1, 5 mmol of 4-diethylaminobenzaldehyde and a few drops of piperidine, and the mixture was subjected to reflux for 6 hrs and the resulting water was removed. The mixture was cooled to room temperature, and then benzene was evaporated. The precipitate was filtered and purified by column chromatography on silica gel with the use of chloroform as an eluate to give 2,3-dicyano-5-hydroxy-5-phenyl-7-methyl-6-[(4-dimethylaminophenyl)methylidene]-4H-1,4-diazepine (5b) in 12% yield. The results of analyses are described as follows.

melting point (decomposition): 180 to 183° C.

NMR δ_{H}(CDCl₃): 13.92 (1H, s, NH), 8.39 (1H, s, OH), 8.07 (2H, broad, phenyl protons), 7.97 (2H, d, J=7, phenyl protons), 7.51 (3H, m, J=7, phenyl protons), 6.74 (2H, d, J=9, phenyl protons), 6.09 (1H, s, CH), 3.49 (4H, q, J=7.2, CH₂), 2.54 (3H, s, CH₃), 1.25 (6H, t, J=7.2, CH₃)

| Elemental analysis | C (%) | H (%) | N (%) |
|---|---|---|---|
| Calculated | 72.97 | 6.12 | 17.02 |
| Found | 72.47 | 6.41 | 16.59 |
| mass spectrum m/e(M⁺): | 411 | | |

### Synthesis Example 8

The object compound 2,3-dicyano-5-hydroxy-5-ethoxy-7-methyl-6-[(4-dimethylaminophenyl)methylidene]-4H-1,4-diazepine (5c) in 23% yield was obtained in the same manner as in Synthesis example 7 with the exception that the compound 3c of Synthesis example 2 was used in lieu of the compound 3b of Synthesis example 1. The results of analyses are described as follows.

melting point (decomposition): 164 to 166° C.

NMR δ_{H}(CDCl₃): 11.97 (1H, s, NH), 8.33 (1H, s, OH), 7.90 (2H, d, J=9.0, phenyl protons), 6.69 (2H, d, J=9.0, phenyl protons), 4.97 (1H, s, CH), 4.25 (2H, q, J=7.2, CH₂), 3.46 (4H, q, J=7.2, 2CH₂), 2.38 (3H, s, CH₃), 1.32 (3H, t, J=7.2, CH₃), 1.23 (6H, t, J=7.2, 2CH₃)

| Elemental analysis | C (%) | H (%) | N (%) |
|---|---|---|---|
| Calculated | 66.47 | 6.64 | 18.46 |
| Found | 66.43 | 6.54 | 17.91 |
| mass spectrum m/e(M⁺): | 379 | | |

### Example 1

The object compound 2,3-dicyano-5-hydroxy-5-dimethylamino-7-methyl-6-[(4-dimethylaminophenyl)methylidene]-4H-1,4-diazepine (5d) in 25% yield was obtained in the same manner as in Synthesis example 7 with the exception that the compound 3d of Synthesis example 3 was used in lieu of the compound 3b of Synthesis example 1. The results of analyses are described as follows.

melting point (decomposition): 155 to 158° C.

NMR δ_{H}(CDCl₃): 13.39 (1H, s, NH), 8.31 (1H, s, OH), 7.95 (2H, broad, phenyl protons), 6.67 (2H, d, J=7.8, phenyl protons), 5.17 (1H, s, CH), 3.44 (4H, q, J=7.2, CH₂), 3.06 (6H, s, NCH₃), 2.39 (3H, s, CH₃), 1.22 (6H, t, J=7.2, CH₃)

| Elemental analysis | C (%) | H (%) | N (%) |
|---|---|---|---|
| Calculated | 66.64 | 6.92 | 22.21 |
| Found | 66.52 | 6.97 | 22.21 |
| mass spectrum m/e(M⁺): | 378 | | |

### Example 2

The object compound 2,3-dicyano-4a-hydroxy-9-methyl-8-(4-diethylaminophenyl)-4H,6H,7H-oxacyclohexano[2,3-e]-1,4-diazepine (5e) in 40% yield was obtained in the same manner as in Synthesis example 7 with the exception that the compound 3e of Synthesis example 4 was used in lieu of the compound 3b of Synthesis example 7. The results of analyses are described as follows.

melting point (decomposition): 184 to 185° C.

NMR δ_{H}(CDCl₃): 11.63 (1H, s, NH), 8.31 (1H, s, OH), 7.91 (2H, broad, phenyl protons), 6.72 (2H, d, J=8, phenyl protons), 4.41 (2H, t, CH₂), 3.44 (4H, q, J=7, CH₂), 3.00 (2H, t, CH₂), 2.43 (3H, s, CH₃), 1.22 (6H, t, J=7, CH₃); δ_{C}(CDCl₃): 172.9 (7-C), 160.5 (2′-C), 151.7 (4′-C), 146.5 (3-C), 122.2 (1′-C), 117.1 (CN), 114.7, 113.5, 112.3 (2-, 6-, and olefinic-C), 111.5 (3′-C), 98.4 (5′-C), 65.4 (OCH₂), 44.7 (NCH₂), 26.3 (OCH₂CH₂), 17.8 (7-Me), 12.6 (NCH₂Me)

| Elemental analysis | C (%) | H (%) | N (%) |
|---|---|---|---|
| Calculated | 66.82 | 6.14 | 18.56 |
| Found | 66.42 | 6.33 | 18.28 |
| mass spectrum m/e(M⁺): | 378 | | |

### Synthesis Example 9

Into a flask provided with Dean-Stark trap were fed 50 ml of benzene, 5 mmol of the compound 4a of Synthesis example 5, 5 mmol of 4-diethylaminobenzaldehyde and a few drops of piperidine, and the mixture was subjected to reflux for 6 hrs and the resulting water was removed. The mixture was cooled to room temperature, and then benzene was evaporated. The precipitate was filtered and purified by column chromatography on silica gel with the use of chloroform as an eluate to give 2,3-dicyano-5-methyl-7-[2-(4-dimethylaminophenyl)ethenyl]-6H-1,4-diazepine (6a) in 50% yield. The results of analyses are described as follows.

melting point (decomposition): over 300° C.

NMR δ_{H}(CDCl₃): 7.45(2H, d, J=8.7, phenyl protons), 7.44 (1H, d, J=15.9, CH), 6.68 (2H, d, J=8.7, phenyl protons), 6.67 (1H, d, J=15.9, CH), 4.57 (1H, broad, CH₂), 3.43 (4H, q, J=7.2, 2CH₂), 1.83 (1H, broad, CH₂), 1.59 (3H, s, CH₃), 1.21 (6H, t, J=7.2, 2CH₃)

| Elemental analysis | C (%) | H (%) | N (%) |
|---|---|---|---|
| Calculated | 72.48 | 6.39 | 21.13 |
| Found | 72.63 | 6.40 | 20.41 |
| mass spectrum m/e(M⁺): | 331 | | |

### Synthesis Example 10

The object compound 2,3-dicyano-5-phenyl-7-[2-(4-dimethylaminophenyl)ethenyl]-6H-1,4-diazepine (6b) in 33% yield was obtained in the same manner as in Synthesis example 9 with the exception that the compound 4b of Synthesis example 6 was used in lieu of the compound 4a of Synthesis example 5. The results of analyses are described as follows.

melting point (decomposition): 198 to 200° C.

NMR δ_{H}(CDCl₃): 7.99 (2H, d, J=8.1, phenyl protons), 7.50 (3H, m, phenyl protons), 7.50 (1H, d, J=15.9, CH), 7.36 (2H, d, J=8.7, phenyl protons), 6.61 (2H, d, J=8.7, phenyl protons), 6.58 (1H, d, J=15.9, CH), 5.30 (1H, broad, CH₂), 3.40 (4H, q, J=7.2, 2CH₂), 1.95 (1H, broad, CH₂) 1.19 (6H, t, J=7.2, 2CH₃)

| Elemental analysis | C (%) | H (%) | N (%) |
|---|---|---|---|
| Calculated | 76.31 | 5.89 | 17.80 |
| Found | 75.78 | 6.04 | 17.27 |
| mass spectrum m/e(M⁺): | 331 | | |

The absorption spectra and the fluorescence spectra of the compounds thus obtained were measured. The results are shown in Table 1. In the Table 1, numbers of compounds represent numbers of compounds obtained by Examples and Synthesis examples. λmax represents the absorption maximum wavelength, and εmax represents the absorption coefficient in the absorption maximum wavelength λmax, and Fmax represents the maximum absorption wavelength of fluorescence in the table.

TABLE 1

| | | | | fluorescence | |
|---|---|---|---|---|---|
| | | | | intensity | Stokes |
| | λ max | | F max | (relative | shift |
| | (nm) | ε max | (nm) | intensity) | (nm) |
| 5b | 529 | 41,000 | 582 | 1420 (4.1) | 53 |
| 5c | 500 | 45,500 | 543 | 600 (1.7) | 43 |
| 5d | 502 | 34,200 | 543 | 350 (1.0) | 41 |
| 5e | 510 | 37,400 | 554 | 730 (2.1) | 44 |
| 6a | 493 | 42,400 | 591 | 1600 (4.6) | 98 |
| 6b | 511 | 35,500 | 632 | 800 (2.3) | 111 |

As clarified in Table 1, any compounds above have large absorption coefficients. Absorption coefficients of compounds having a group (dimethyl amino group of the compound 5d, phenyl groups of the compound 5b and 6b) which a freedom degree of rotation is small as R² groups, are smaller than those of other compounds.

Moreover, in the fluorescence spectrum, although the absorption wavelength of the compound of the formula (II) is shorter than that of the compound of the formula (I), the compound of the formula (II) emits yellow to orange of which the wavelength is relatively long, since the compound of the formula (II) is easier to make a conjugate system. Particularly, in the case where R² of the compound of the formula (II) is a phenyl group, the fluorescence in the longest wavelength region emits.

### Examples 3 and 4

Each (1.1 mg) of the compound 5d and 5e obtained in Example 1 and 2 was dissolved in 50 ml of 1,2-dichloroethane respectively. To 15 ml of the solution was added 5 ml of 1,2-dichloroethane, and N,N′-diphenyl-N,N′-bis(3-methylphenyl)-1,1′-biphenyl-4,4′-diamine (TPD), 2-(4-biphenyl)-5-(4-tert-butylphenyl)-1,3,4-oxadiazole (PBD), and polymethyl methacrylate (PMMA) as a binder were further added in the proportion described in the Table 2 to give a coating solution.

TABLE 2

| | Example 3 | Example 4 |
|---|---|---|
| TPD (mg) | 49.4 | 50.6 |
| PBD (mg) | 99.6 | 100.6 |
| PMMA (mg) | 150.4 | 150.4 |

After forming a film (coat) of indium-tin-oxide (ITO) on a glass substrate, the coating solution was applied on the ITO film by spin coating (coating conditions: 1000 rpm, 10 sec for 5d and 2000 rpm, 10 sec for 5e) to form a light-emitting layer of about 0.1 μm thick. Thereafter, on the light-emitting layer, an Al/Li electrode of 200 nm thick (manufactured by Kojundo Kagaku, K.K.; Li content 0.78% by weight) was made by vacuum deposition to give an organic electroluminescence device.

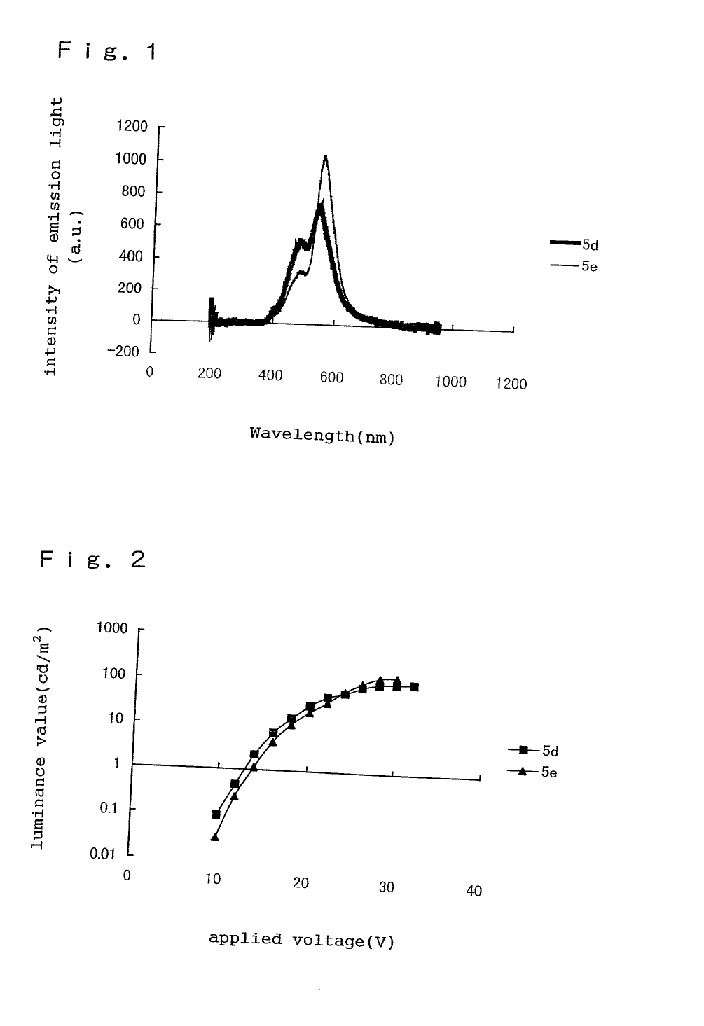

With the ITO electrode of the organic electroluminescence device obtained as anode and the AL/Li electrode as cathode, a voltage was applied between the both electrodes in the atmosphere thereby to make the device emit light. The emission spectrum under applied voltage of 22V was measured. The emission spectrum is shown in FIG. 1. A graph showing the value of luminance versus voltage applied is shown in FIG. 2.

What is claimed is:

1. An azepine compound represented by the following formula (I) or (II): 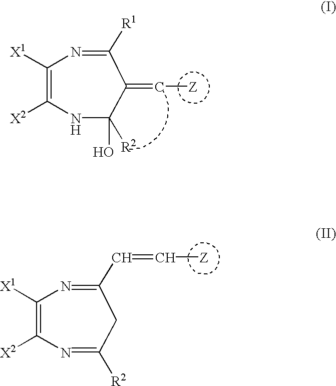
wherein X¹ and X² are the same or different, each representing an electron attractive group; R¹ represents a hydrogen atom, an alkyl group, an aryl group, an aralkyl group, or an alkoxy group; R² represents an amino group or an N-substituted amino group, or, in the formula (I), R² bonds a carbon atom adjacent to a ring Z to form a ring; and the ring Z represents a hydrocarbon ring which may have a substituent or a heterocycle which may have a substituent.

2. An azepine compound according to claim 1, wherein at least one of X¹ and X² is a cyano group.

3. An azepine compound according to claim 1, wherein R² is an N- mono- or diC₁₋₄alkylamino group.

4. An azepine compound according to claim 1, wherein R² bonds a carbon atom adjacent to the ring Z to form a 4- to 8-membered heterocycle.

5. An azepine compound according to claim 1, wherein the hydrocarbon ring or the heterocycle of the ring Z is an aromatic ring.

6. An azepine compound according to claim 1, wherein X¹ and X² each is a cyano group, R¹ is a C1-4alkyl group or a C6-12aryl group, R² is an amino group or an N-mono- or diC₁₋₄alkylamino group, or R² bonds a carbon atom adjacent to the ring Z to form a5- or 6-membered heterocycle, and Z is an aromatic ring having a substituent.

7. An azepine compound according to claim 1, which is capable of emitting fluorescent light by being irradiated with light.

8. An azepine compound according to claim 1, which is capable of emitting light by the action of an electric field.

9. A process for producing an azepine compound represented by the following formula (I) or (II): 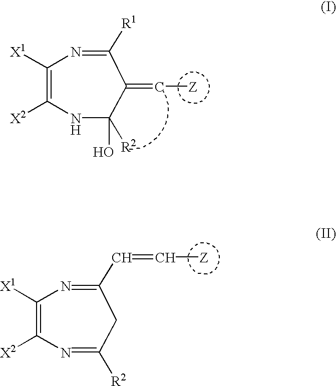
wherein X¹ and X² are the same or different, each representing a hydrogen atom, an alkyl group, or an electron attractive group and at least one of which being an electron attractive group; R¹ represents a hydrogen atom, an alkyl group, an aryl group, an aralkyl group, or an alkoxy group; R² represents an amino group or an N-substituted amino group, or, in the formula (I), R² bonds a carbon atom adjacent to a ring Z to form a ring; and the ring Z represents a hydrocarbon ring which may have a substituent or a heterocycle which may have a substituent
which comprises reacting a compound represented by the following formula (Iₐ) or (IIₐ): 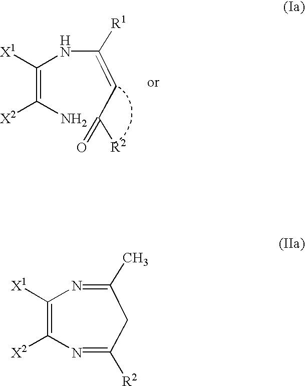
wherein R² represents an amino group or an N-substituted amino group, or, in the formula (Iₐ), R² bonds a carbon atom in β-position to R² to form a ring, and the X¹, X², and R¹ have the same meanings as defined above.
with a compound represented by the following formula (III): 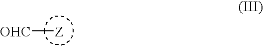
wherein the ring Z has the same meaning as defined above.

10. An organic electroluminescence device, which comprises a pair of electrodes and an organic layer interposed therebetween, wherein the organic layer comprises a compound represented by the formula (I) or (II) recited in claim 1.

11. An organic electroluminescence device according to claim 10, wherein the organic layer has a light-emitting layer comprising a compound represented by the formula (I) or (II).

12. An organic electroluminescence device according to claim 10, wherein the organic layer has (1) a single layer structure composed of a light-emitting layer having at least one function selected from an electron-transporting function and a hole-transporting function, or (2) a layered structure composed of a layer having at least one function selected from an electron-transporting function and a hole-transporting function, and a light-emitting layer.
